(19) United States
(12) Patent Application Publication
(10) Pub. No.: US 2006/0029589 A1
(43) Pub. Date: Feb. 9, 2006

(54) Induction of heterozygous FV Leiden carrier status to reduce mortality in sepsis and to prevent organ damage caused by inflammation and/or ischemia-reperfusion injury

(76) Inventor: Hartmut Weiler, Milwaukee, WI (US)

Correspondence Address: QUARLES & BRADY LLP, 411 E. WISCONSIN AVENUE, SUITE 2040, MILWAUKEE, WI, 53202-4497, US

(21) Appl. No.: 11/141,660

(22) Filed: May 31, 2005

Related U.S. Application Data
(60) Provisional application No. 60/575,943, filed on Jun. 1, 2004.

(51) Int. Cl. A61K 38/48

(52) U.S. Cl. 424/94.63

(57) ABSTRACT

A method of treating a disease is disclosed. In one embodiment, the method comprises administering to a patient an effective amount of factor V protein or fragment of factor V protein, wherein the amount of protein is sufficient to alleviate or prevent disease symptoms and wherein the protein is resistant to inactivation by APC.

### CROSS-REFERENCE TO RELATED APPLICATION

This application claims priority to U.S. provisional application 60/575,943, filed Jun. 1, 2004, incorporated by reference as if fully set forth herein.

### STATEMENT REGARDING FEDERALLY SPONSORED RESEARCH OR DEVELOPMENT

### BRIEF DESCRIPTION OF THE SEVERAL VIEWS OF THE DRAWINGS

FIG. 1 is a graph of survival analysis for normal, heterozygous and homozygous FV Leiden mice infected with Staphylococcus aureus.

### BACKGROUND OF THE INVENTION

The blood coagulation factor V (FV) is a pivotal enzyme cofactor of the enzymatic blood coagulation reaction (Nicolaes, G. A. and B. Dahlback, Arterioscler. Thromb. Vasc. Biol. 22 (4):530-538, 2002). It circulates in blood at a concentration of approximately 0.007 g/L. The activated form of FV is an essential cofactor for the factor Xa-dependent generation of the central coagulation protease, thrombin.

The protein C-pathway is a naturally occurring anticoagulant mechanism that curbs the excessive formation of thrombin. The anticoagulant capacity of this pathway is based on the augmented formation of activated protein C (APC) by the thrombomodulin-thrombin complex in response to initiation of the coagulation reaction. APC then degrades activated coagulation factors Va and Villa by partial proteolysis. APC cleaves coagulation FV and activated coagulation FV at several positions (Arg306, Arg506, and Arg679). Proteolysis at Arg506 partially abolishes the ability of FV to participate in the generation of thrombin, and protein S-supported cleavage at Arg306 is required for complete inactivation of activated FV. Proteolysis of intact FV at Arg506 generates a FV derivative that supports the degradation of FVI II by APC.

A common polymorphism in the FV gene is FV Leiden. This mutation results in an amino acid substitution in the FV protein (Arg506→Gln) that renders activated FV partially resistant to inactivation by APC (Dahlback, B., Semin. Hematol. 34 (3):217-234, 1997). The FV Leiden mutation therefore disrupts the ability of the protein C pathway to curb thrombin generation, and heterozygous carriers of the FV Leiden allele are at increased risk to develop venous thrombosis.

The prevalence of the FV Leiden polymorphism in the general population reaches between 2 and 15% percent, and up to 60% in patients with venous thromboembolism (Rosendaal, F. R., Lancet 353 (9159):1167-1173, 1999). The astonishingly high frequency of FV Leiden carriers has given rise to speculations that the FV Leiden mutation conveys some kind of survival advantage, which might have exerted a positive selection pressure to maintain this polymorphism in the general gene pool.

### BRIEF SUMMARY OF THE INVENTION

It has been recently discovered (a) that mice carrying one allele of the coagulation factor V Leiden gene variant and one allele of the normal factor V gene (heterozygous FV Leiden carriers) are indeed protected from the lethal consequences of endotoxin-induced septicemia, respectively, and (b) that heterozygous carrier status for the FV Leiden allele also improved the odds of survival of human patients with severe sepsis approximately three-fold, as compared to subjects carrying two normal factor V alleles (Kerlin, et al., Blood, November 2003 [102]9, 3085-3092, incorporated by reference).

Heterozygous carriers of the Leiden allele may also benefit in other settings, such as a reduction of bleeding complications caused by surgery (Donahue, et al., Circulation 107 (7):1003-1008, 2003) or menstruation or childbirth (Lindqvist, P. G., et al., Thromb. Haemost. 86 (4):1122-1123, 2001).

Follow-up experiments (reported below) demonstrated that heterozygous carrier status of the Leiden allele protected mice against lethality after intraperitoneal administration of LD50 of gram-positive bacterial pathogen such as Staphylococcus aureaus. These findings show that heterozygous carrier status for FV Leiden is beneficial in the host response to bacterial infection and ameliorates lethality from severe inflammatory responses in the absence of bacteria.

In one embodiment, the present invention is a method of treating sepsis comprising administering to a sepsis patient an effective amount of altered factor V protein, wherein the amount of protein is sufficient to alleviate or prevent sepsis symptoms and wherein the alteration renders the protein resistant to inactivation by APC. A preferred altered Factor V protein is the Factor V Leiden protein.

In another embodiment, the present invention is a method of preventing organ damage caused by inflammation comprising administering to an inflammation patient an effective amount of altered factor V protein, wherein the amount of protein is sufficient to alleviate inflammation symptoms and wherein the alteration renders the protein resistant to inactivation by APC.

In another embodiment, the present invention is a method of treating ischemia re-perfusion injury by administering to a reperfusion injury patient an effective amount of altered factor V protein, wherein the amount of protein is sufficient to alleviate injury symptoms and wherein the alteration renders the protein resistant to inactivation by APC.

Other embodiments and advantages of the present invention will become apparent after review of the specification, claims and drawings.

### DESCRIPTION OF THE INVENTION

In one embodiment, the present invention is a method of treating a disease patient, preferably treating septic patients, by artificially inducing a functional factor V Leiden carrier status in the patient. In one preferred method, this status is achieved by infusing, or otherwise inducing production, of the factor V Leiden protein or a protein fragment to replicate the beneficial effects known and as yet unknown experienced by heterozygous carriers of the factor V Leiden allele.

In general, this may be achieved by measuring and monitoring with existing methods the plasma level of functional FV in the patient and infusing the amount of functional FV Leiden protein needed to at least equal the existing plasma level (±10%) of endogenous FV. One would preferably want to have the amount of FV Leiden equal the amount of endogenous FV, however, a lesser or greater amount of FV Leiden would also be suitable. Specifically, if the mechanism of action requires the matching of FV with FV Leiden, an excess of FV Leiden would not be necessary but would still be a workable embodiment of the present invention. Factor V Leiden protein, combined with a pharmaceutical carrier, may be preferably administered through continuous infusion over prolonged periods of time (2-10 days). In another embodiment of the invention, the Factor V protein may be administered via gene therapy methods.

Factor V Leiden protein for this purpose may be obtained by expression of recombinant protein in eukaryotic cells or by purification from plasma from heterozygous carriers. One could obtain suitable Factor V Leiden protein or a gene encoding Factor V Leiden protein in several manners know to one of skill in the art. One could review references disclosing FV Leiden, such as Bertina, R. M., et al., Nature 369 (6475):64-67, 1994 (incorporated by reference), to obtain information useful in obtaining the FV Leiden gene or protein.

We envision that fragments of Factor V Leiden protein would be sufficient for the present invention. A suitable fragment of Factor V Leiden protein would contain the site resistant to proteolysis by APC. One could preferably test suitable fragments by the method presented below in the example. One would infuse normal mice with the fragment and determine whether the mouse was protected at a level within 10% of the heterozygous mouse in the Example.

In another embodiment, portions or modified forms of the factor V protein, which replicate the beneficial effects of the Factor V Leiden protein, may be used. Such forms may include FV variants that are resistant to proteolysis by APC at APC target motifs (cleavage sites) other than Arg506, such as the FV Hongkong and Cambridge variants, or modified forms, such as B-domain-less FV. The FV HongKong is Arg306→Gln (or 306Q). The FV Cambridge is Arg306→Thr. (See Chan, W. P., et al., Blood 91:1135-1139, 1998; Williamson, D., et al., Blood 91:1140-1144, 1998) The third APC cleavage site is Arg679 (van der Neut Kolfshoten, M., et al., J. Biol. Chem. 279:6567-6575, 2004).

As with FV Leiden, the protein(s) or protein fragments will be administered to achieve circulating blood levels in a range similar to that of FV present in the patient (as measured at the time of analysis).

In another embodiment, induction of functional Leiden status by the above means will be administered to support and enhance therapy with other agents. As one, but not the only possibility, we envision co-infusion of human protein C zymogen, especially in patients with significantly reduced protein C levels due to consumption (Liaw, P. C., et al., Blood 104 (13):3958-3964, 2004). We also envision that co-infusion of APC-resistant FV with APC into septic patients will (a) enhance the therapeutic efficacy of treatment, as compared to APC alone, and will (b) prevent the bleeding complications observed resulting from APC treatment.

In another embodiment of the present invention, one would artificially induce a functional Factor V Leiden carrier status to reduce mortality or morbidity of patients with organ damage caused by inflammation and/or ischemia re-perfusion injury.

The potential beneficial effects of the induced Factor V Leiden status may include, but are not limited to
reducing mortality of patients with severe sepsis and multi-organ failure,
reducing morbidity of patients with sepsis or severe sepsis,
preventing multi-organ failure in septic patients,
preventing onset of severe sepsis in patients at risk to suffer multi-organ failure,
enhancing preservation and recovery of organ function after ischemic reperfusion injury as it may occur in stroke or myocardial infarction, and
limiting organ damage caused by inflammatory mechanisms, including cytokine- and complement-dependent mechanisms.

Proposed Mechanism of Action:

Several candidate mechanisms acting alone or in synergy might account for the beneficial effect of heterozygous FV Leiden carrier status: FV Leiden carriers show enhanced thrombin formation, and enhanced thrombin formation may in turn lead to enhanced protein C activation (Kerlin, 2003, in Supra). In effect, the FV Leiden mutation would thereby replicate the known and as yet unknown effects of APC. The known effects of APC include, but are not limited to counteracting the loss of blood pressure in sepsis, reducing the magnitude of cytokine responses by various immune cells to inflammatory stimuli, supporting vascular endothelial cell survival by inducing a specific gene expression profile in endothelial cells, and coordinating fibrinolytic system activity (Esmon, C. T., Ann. Med. 34 (7-8):598-605, 2002). Enhanced thrombin generation may also support the inhibition of complement-induced organ damage via the thrombin-dependent activation of procarboxypeptidases, which inactivate critical complement factors and suppress fibrinolysis (Esmon, C. T., supra, 2002; Laudes, I. J., et al., Am. J. Pathol. 160 (5):1867-1875, 2002).

In acute inflammation, the plasma concentration of procarboxypeptidases is increased, while levels of protein C and FV are decreased. This imbalance might favor activation of procarboxypeptidases over protein C activation, thereby more effectively inhibiting complement to achieve better protection against fatal organ damage. An important, different mechanism is likely mediated by the augmented generation of fibrin and the increased inhibition of fibrinolysis in heterozygous Leiden carriers, which may augment bacterial clearance and inhibit bacterial dissemination in a fibrin-dependent manner. Together, our findings suggest that heterozygous FV Leiden status supports several distinct pathways with beneficial outcome on the host response to bacterial infection and severe inflammatory disease. Thereby, FV Leiden carrier status, whether genetically based or artificially induced by infusion of the gene product, may exert a larger benefit than predicted from targeting only one pathway. It is noteworthy in this respect that the benefit derived from heterozygous FV Leiden status in severe sepsis is indeed approximately 2-fold better than that derived from infusion of APC. In addition, the FV Leiden protein may also have as yet undiscovered activities not shared by the normal FV protein, and the beneficial effect of the FV Leiden protein might be based on such a gain of function.

### EXAMPLE

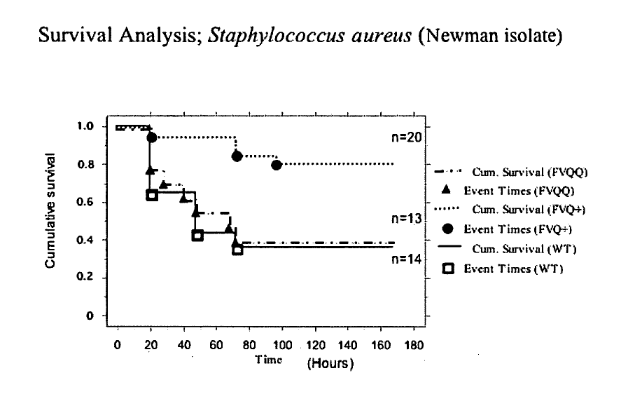

FIG. 1 demonstrates an experiment showing survival analysis of normal, heterozygous and homozygous FV Leiden mice infected with Staphylococcus aureus. Normal (wt), heterozygous (fVQ/+), and homozygous FV Leiden mice (fVQQ) were infected with 2×10⁸ S. aureus bacteria by intraperitoneal injection. This dose was determined in pilot experiments to cause the death of approximately 50% of normal mice. FVQ/+mice have a significant survival advantage over wildtype and homozygous fVQQ mice (P=0.008 Mantel-Cox Logrank). These data mirror precisely the survival benefit seen in LPS-induced septicemia, where homozygous fVQQ mice and wildtype mice showed identical survival, but fVQ/+mice were significantly protected.

I claim:

1. A method of treating a disease comprising administering to a patient an effective amount of factor V protein or fragment of factor V protein, wherein the amount of protein is sufficient to alleviate or prevent disease symptoms and wherein the protein is resistant to inactivation by APC.

2. The method of claim 1 wherein the disease is selected from the group consisting of sepsis, organ damage caused by inflammation and ischemia reperfusion injury.

3. The method of claim 1 wherein the Factor V protein is Factor V Leiden.

4. The method of claim 1 wherein the treatment is between 2-10 days.

5. The method of claim 1 wherein the protein is obtained by expression of recombinant protein.

6. The method of claim 1 wherein the protein is obtained by purification of plasma from heterozygous carriers.

7. The method of claim 1 additionally comprising the step of treating the patient with human protein C.

8. A method of treating sepsis comprising administering to a sepsis patient an effective amount of altered factor V protein or protein fragment, wherein the amount of protein is sufficient to alleviate or prevent sepsis symptoms and wherein the alteration renders the protein resistant to inactivation by APC.

9. The method of claim 8 wherein the protein is Factor V Leiden protein.

10. A method of alleviating organ damage caused by inflammation comprising administering to an inflammation patient an effective amount of altered factor V protein or protein fragment, wherein the amount of protein is sufficient to alleviate inflammation symptoms and wherein the alteration renders the protein resistant to inactivation by APC.

11. The method of claim 10 wherein the protein is Factor V Leiden protein.

12. A method of treating ischemia re-perfusion injury by administering to a reperfusion injury patient an effective amount of altered factor V protein or protein fragment, wherein the amount of protein is sufficient to alleviate or preventing injury symptoms and wherein the alteration renders the protein resistant to inactivation by APC.

13. The method of claim 12 wherein the protein is Factor V Leiden protein.
